(19) United States
(12) Patent Application Publication
(10) Pub. No.: US 2002/0147448 A1
(43) Pub. Date: Oct. 10, 2002

(54) Bipolar symmetrical rotational electrode

(76) Inventors: Steven Fodor, Walden, NY (US); David Kim, Los Angeles, CA (US)

Correspondence Address: Steven Fodor, 2274 Albany Post Rd., Walden, NY, 12586, US

(21) Appl. No.: 09/828,687

(22) Filed: Apr. 9, 2001

(51) Int. Cl. A61B 18/18

(52) U.S. Cl. 606/48

(57) ABSTRACT

Bipolar Symmetrical Rotational Electrode characterized by two separated wires which cut and coagulate in bipolar electrosurgical mode to take a biopsy.

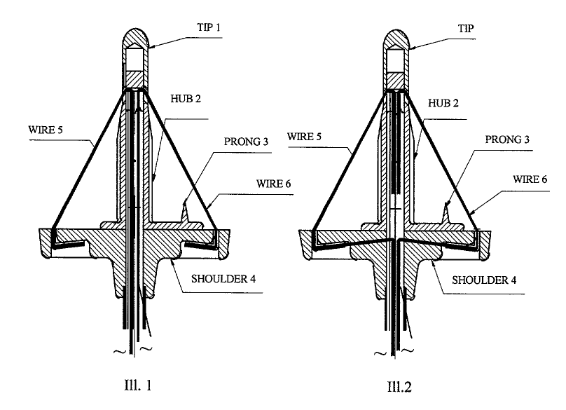

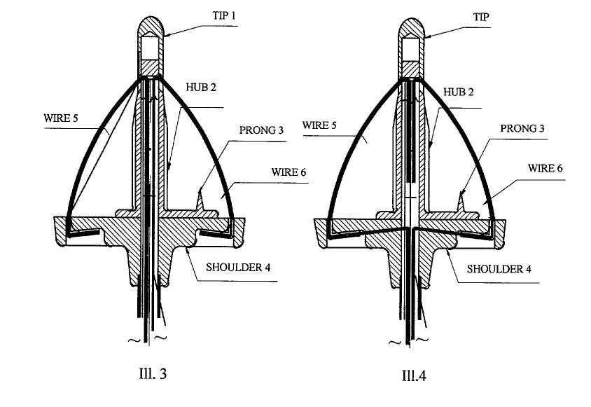

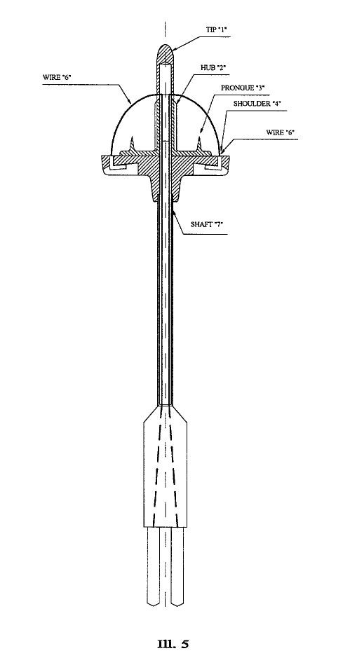

### CROSS REFERENCE TO RELATED APPLICATIONS

Kim Cone Biopsy Instrument U.S. Pat. No. 5,676,663.

### BACKGROUND OF INVENTION

A cancerous cervical lesion can be removed by various techniques: a cold knife cutting, usually complicated by intensive bleedingb.triangle shaped Olsen or Greenwald type electrosurgical conization electrode, with difficulties to control the biopsyc.LLETZ electrodes, producing only slices of tissued.KIM rotational hub cone biopsy electrosurgical electrode (U.S. Pat. No. 5,676,663), with a high degree of safety. Like all cone biopsy electrodes, the KIM electrode is rotated 360 degrees and the monopolar high frequency blend current, along the conductive wire, will cut and coagulate the tissue at the same time. The current will flow between the monopolar instrument and the large surface dispersive conductive pad. All monopolar techniques require the use of an additional dispersive conductive pad, also described as grounding pads. The KIM electrode will produce a perfectly shaped tissue cone. The KIM electrode has a stabilizing hub with sharp prongs to stabilize the cervical tissue during the rotating cutting.

### SUMMARY OF INVENTION

We have invented a bipolar rotational electrode very similar in all other features to the KIM electrode. The bipolar instrument holds two symmetrically arranged separate wires on each side. The bipolar electrosurgical energy is separately connected to the wires. Once in contact with the tissue, the wires will perform the cutting and coagulation, as current flows between the wires. To achieve the cone biopsy, this bipolar instrument must be rotated only 180 degrees. The family of the instruments is made in a range of sizes to fit the requirement of surgery. The bipolar procedure is safer than the monopolar one, since there is no grounding pad. There is no danger that burns will occur at the grounding pads. The safety of the bipolar procedure is superior to the monopolar one.

### BRIEF DESCRIPTION OF DRAWINGS

1. Bipolar symmetrical rotational electrode with two symmetrically arranged straight separate cutting wires to perform a biopsy in bipolar mode.

2. Bipolar symmetrical rotational electrode with two symmetrically arranged curved separate cutting wires to perform a biopsy in bipolar mode.

3. Bipolar symmetrical rotational electrode combined with a stabilizing core and prongs to improve the safety of a biopsy.

4. Bipolar symmetrical rotational electrode with a rotation knob for easy manual rotation. Bipolar symmetrical rotational electrode with symmetrical wires to perform an excision, resection or cutting.
